SEQUENCE LISTING (1 sequence)

SEQ ID NO: 1 - protein, 675 aa, artificial
  synthetic_sequence
MDTKSILEELLLKRSQQKKKMSPNNYKERLFVLTKTNLSYYEYDKMKRGSRKGSIEIKKIRCVEKVNLEEQTPVERQYPFQIVYKDGLLYVYASNEESRSQWLKALQKEIRGNPHLLVKYHSGFFVDGKFLCCQQSCKAAPGCTLWEAYANLHTAVNEEKHRVPTFPDRVLKIPRAVPVLKMDAPSSSTTLAQYDNESKKNYGSQPPSSSTSLAQYDSNSKKIYGSQPNFNMQYIPREDFPDWWQVRKLKSSSSSEDVASSNQKERNVNHTTSKISWEFPESSSSEEEENLDDYDWFAGNISRSQSEQLLRQKGKEGAFMVRNSSQVGMYTVSLFSKAVNDKKGTVKHYHVHTNAENKLYLAENYCFDSIPKLIHYHQHNSAGMITRLRHPVSTKANKVPDSVSLGNGIWELKREEITLLKELGSGQFGVVQLGKWKGQYDVAVKMIKEGSMSEDEFFQEAQTMMKLSHPKLVKFYGVCSKEYPIYIVTEYISNGCLLNYLRSHGKGLEPSQLLEMCYDVCEGMAFLESHQFIHRDLAARNCLVDRDLCVKVSDFGMTRYVLDDQYVSSVGTKFPVKWSAPEVFHYFKYSSKSDVWAFGILMWEVFSLGKQPYDLYDNSQVVLKVSQGHRLYRPHLASDTIYQIMYSCWHELPEKRPTFQQLLSSIEPLREKDKH

(19) United States
(12) Patent Application Publication
(10) Pub. No.: US 2014/0363820 A1
(43) Pub. Date: Dec. 11, 2014

(54) METHOD FOR DETECTING AND EXAMINING TRAUMATIC BRAIN INJURY IN VITRO

(71) Applicants: Kai-yun CHEN, Taipei (TW); Chung-che Wu, Taipei (TW); Yung-hsiao Chiang, Taipei (TW)

(72) Inventors: Kai-yun CHEN, Taipei (TW); Chung-che Wu, Taipei (TW); Yung-hsiao Chiang, Taipei (TW)

(73) Assignee: Taipei Medical University, Taipeu (TW)

(21) Appl. No.: 14/304,999

(22) Filed: Jun. 16, 2014

Related U.S. Application Data
(63) Continuation of application No. 13/543,831, filed on Jul. 8, 2012, now abandoned.

(30) Foreign Application Priority Data
Feb. 17, 2012 (TW) 101105331

(51) Int. Cl. C12Q 1/68

(52) U.S. Cl. CPC C12Q 1/6881; C12Q 2600/158
USPC 435/6.12

(57) ABSTRACT

The present invention discloses a method for detecting and examining traumatic brain injury (TBI) in vitro. The method is according to the principle of expression of Etk/Bmx mRNA which is correspondingly increased when TBI occurs, so that the Etk/Bmx mRNA is defined as a quantification reference indicator specific for neurological injury degree of TBI. Thus, the method can be used to detect the expression of the Etk/Bmx mRNA for examining and evaluating the neurological injury degree of TBI occurred due to an external impact.

### CROSS REFERENCE TO RELATED APPLICATIONS

This application is a continuation application of U.S. patent application Ser. No. 13/543,831, filed on Jul. 8, 2012, which claims a priority from Taiwan Patent Application No. 101105331 filed Feb. 17, 2012, now issued Taiwan Pat. No. 1-429908, the disclosure of which is incorporated herein by reference.

### FIELD OF THE INVENTION

The present invention relates to a method for detecting and examining traumatic brain injury (TBI) in vitro, and more particularly to a method for detecting and examining TBI in vitro, in which the expression of Etk/Bmx mRNA of a sample from a traumatic brain tissue of a patient after occurring TBI due to an external impact is used as a quantification reference indicator for determining a neurological injury degree of TBI in vitro.

### BACKGROUND OF THE INVENTION

Traumatic brain injury (TBI) occurs due to external impact, with or without serious intracranial hemorrhage or fracture of skull. According to the different degree of impact force, TBI is sorted into mild, moderate or severe degree. The head injuries with normal intracranial hemorrhage and fracture of skull are easy to be diagnosed as the TBI from the head appearance of a patient. However, according to clinical statistics, about 40 to 50 percent of patients with mild impact of brains and even without intracranial hemorrhage or fracture of skull would occur secondary neurologic injury or neurological disorder within 1 to 3 months. Furthermore, about 25 percent of patients with mild impact would occur related symptoms after one year.

Recently, the most common diagnostic tools for clinically evaluating injury degree of TBI are computed tomography (CT) and magnetic resonance imaging (MRI), but these diagnostic tools only have low sensitivity and low specificity for diffuse brain injury or moderate brain injury. Meanwhile, the inspection thereof is time-consuming and the equipment cost is high. Further, the recently national and international publications indicated that specific biomarkers can be typically detected in blood serum or cerebrospinal fluid (CSF) of patients with traumatic brain injury, ischemic stroke or others acute brain injury, wherein the biomarkers include the protein expression of SBPD120, SBPD145, SBPD150, MAP2, MBP, NSE, UCH-L1, S100β and GFAP. However, disadvantages of these biomarkers are that some protein molecules lack absolute sensitivity or only have low specificity for brain tissues, and thus it is difficult to be used as a single indicator to clinically evaluate injury degree of TBI.

For example, SBDP120 and SBDP145 proteins are expressed in various tissues, wherein these proteins only have insufficient specificity and are limited to detect the CSF difficulty obtained from patients; the expression of NSE protein could be an evaluation of neurologic injuries in brain, but NSE is too slow to eliminate. Thus, it is also difficult to distinguish if the expression is generated from primary or secondary injuries. Moreover, the hemolysis may induce NSE to be released into blood, so as to effect on the result in diagnosis.

Furthermore, GFAP protein is a biomarker expressing after the neuroglia cells are injured and corresponding to inflammation response. The expression of GFAP is increased significantly and easily detected when TBI occurred. However, except for the cerebrum, if the central nervous system (CNS) is injured, it could also induce to increase the expression of GFAP protein. Thus, this biomarker can not be used as the TBI biomarker with high specificity. Moreover, the disadvantage of S100β protein is that it is associated with the permeability of blood-brain barrier (BBB). Once the permeability changed, the expressive levels of S100β is changed even without TBI event.

According to statistics of the Department of Health (DOH) in Taiwan, the accident events are the top ten causes of death, wherein TBI caused by vehicle and fall accidences are major factors of accidental death. Further, the promotion of bicycle sport in the world, the World Health Organization (WHO) made a declaration to member countries for pay more attention to the medical payments and the issues of head injury in 2006. Therefore, for TBI, it would be especially helpful for the diagnosis and therapy of TBI if a biomarker can be used to predict neurological condition of TBI during clinical diagnosis and research and can be simultaneously used as a surrogate endpoint for predicting therapeutic effect.

As a result, it is necessary for biomedical researchers to think how to develop a method for detecting and examining of neurological injury of TBI, in order to substantially solve the existing problems of the traditional techniques, as described above.

### SUMMARY OF THE INVENTION

Therefore, the present invention discloses a method for detecting and examining traumatic brain tissue (TBI) in vitro, to solve relative problems in traditional techniques of biomarkers.

A primary object of the present invention is to provide a method for detecting and examining TBI in vitro, which is according to the principle of expression of Etk/Bmx protein or mRNA which is correspondingly increased when TBI occurs, so that the Etk/Bmx protein or mRNA is defined as a quantification reference indicator for determining a neurological injury degree of TBI in vitro.

To achieve the above object, the present invention provides a method for detecting and examining TBI in vitro, which comprises steps of:

(a) sampling a sample from a traumatic brain tissue of an animal after occurring TBI due to an external impact;

(b) examining the sample for detecting an expression of Etk/Bmx protein expressed due to TBI, wherein amino acid sequences of Etk/Bmx protein are SEQ ID NO:1; and

(c) defining the expression of Etk/Bmx protein as a quantification reference indicator of TBI injury degree for examining and evaluating a neurological injury degree of TBI due to the external impact;

wherein the amino acid sequences of Etk/Bmx protein is SEQ ID NO:1, comprising:

| | |
|---|---|
| MDTKSILEEL LLKRSQQKKK MSPNNYKERL | 30 |
| FVLTKTNLSY YEYDKMKRGS RKGSIEIKKI | 60 |
| RCVEKVNLEE QTPVERQYPF QIVYKDGLLY | 90 |
| VYASNEESRS QWLKALQKEI RGNPHLLVKY | 120 |
| HSGFFVDGKF LCCQQSCKAA PGCTLWEAYA | 150 |
| NLHTAVNEEK HRVPTFPDRV LKIPRAVPVL | 180 |
| KMDAPSSSTT LAQYDNESKK NYGSQPPSSS | 210 |
| TSLAQYDSNS KKIYGSQPNF NMQYIPREDF | 240 |
| PDWWQVRKLK SSSSSEDVAS SNQKERNVNH | 270 |
| IISKISWEFP ESSSSEEEEN LDDYDWFAGN | 300 |
| ISRSQSEQLL RQKGKEGAFM VRNSSQVGMY | 330 |
| TVSLFSKAVN DKKGTVKHYH VHTNAENKLY | 360 |
| LAENYCFDSI PKLIHYHQHN SAGMITRLRH | 390 |
| PVSTKANKVP DSVSLGNGIW ELKREEITLL | 420 |
| KELGSGQFGV VQLGKWKGQY DVAVKMIKEG | 450 |
| SMSEDEFFQE AQTMMKLSHP KLVKFYGVCS | 480 |
| KEYPIYIVIE YISNGCLLNY LRSHGKGLEP | 510 |
| SQLLEMCYDV CEGMAFLESH QFIHRDLAAR | 540 |
| NCLVDRDLCV KVSDFGMTRY VLDDQYVSSV | 570 |
| GTKFPVKWSA PEVFHYFKYS SKSDVWAFGI | 600 |
| LMWEVFSLGK QPYDLYDNSQ VVLKVSQGHR | 630 |
| LYRPHLASDT IYQIMYSCWH ELPEKRPTFQ | 660 |
| QLLSSIEPLR EKDKH | 675. |

In one embodiment of the present invention, the sample in the step (a) is brain tissue, cerebrospinal fluid or blood serum, which is sampled from the animal.

In one embodiment of the present invention, the TBI of the animal due to the external impact in the step (a) is a closed-type TBI or a punctured-type TBI.

In one embodiment of the present invention, the sample of Etk/Bmx protein in the step (b) is labeled by at least one fluorescent marker for detecting the expression of Etk/Bmx protein.

In one embodiment of the present invention, at least one specific primary antibody of Etk/Bmx protein is used in the step (b) for examining the sample.

In one embodiment of the present invention, at least one secondary antibody specific for the primary antibody is used in the step (b) for examining the primary antibody.

In one embodiment of the present invention, the secondary antibody is modified by a specific functional group for colorimetric assay, radiometric assay or fluorescent assay.

In one embodiment of the present invention, ELISA, immunoradioassay, immunofluorescent assay or Western blotting is used in the step (b) for detecting the expression of Etk/Bmx protein.

In one embodiment of the present invention, RT-PCR or Q-PCR is used in the step (b) for detecting the expression of Etk/Bmx mRNA.

### DESCRIPTION OF THE DRAWINGS

FIGS. 1A, 113 and 1C are autoradiograms and statistic graphs of the increasing expressions of Etk/Bmx mRNA and protein of brain tissues after TBI experiments by Western blotting according to a preferred embodiment of the present invention;

FIGS. 2A, 2B and 2C are autoradiograms and statistic graphs of the expressions of Etk/Bmx and S100β protein of brain tissues after TBI experiments by Western blotting and frozen section according to the preferred embodiment of the present invention;

FIGS. 3A and 313 are autoradiograms and statistic graphs of the expressions of Etk/Bmx and GFAP protein of brain tissues in different time after TBI experiments by Western blotting according to the preferred embodiment of the present invention;

FIGS. 4A, 4B and 4C are photographs of frozen section of brain tissues after TBI experiments by immunofluorescent staining to the preferred embodiment of the present invention; and

FIG. 5 is an autoradiogram of various protein expressions of brain tissues after TBI experiments by Western blotting according to the preferred embodiment of the present invention.

### DETAILED DESCRIPTION OF THE PREFERRED EMBODIMENTS

The structure and the technical means adopted by the present invention to achieve the above and other objects can be best understood by referring to the following detailed description of the preferred embodiments and the accompanying drawings. Furthermore, directional terms described by the present invention, such as upper, lower, front, back, left, right, inner, outer, side, longitudinal/vertical, transverse/horizontal, and etc., are only directions by referring to the accompanying drawings, and thus the used directional terms are used to describe and understand the present invention, but the present invention is not limited thereto.

The present invention disclosed a method for detecting and examining TBI in vitro. According to the principle of an increased expression of Etk/Bmx of a sample from a traumatic brain tissue of a patient after TBI occurs is corresponding to TBI, so that Etk/Bmx can be defined as a quantification reference indicator for determining a neurological injury degree of TBI in vitro, wherein Etk means epithelial/endothelial tyrosine kinase and Bmx means Tec kinase bone marrow tyrosine kinase gene in chromosome X. Therefore, the method of the present invention is used to evaluate the expression of Etk/Bmx due to TBI after an external impact occurs, in order to evaluate the neurological injury degree of TBI in vitro. Now, the present invention is described hereinafter by the experiment of simulating TBI in animal model of laboratory studies, in order to detect whether the increased expression of Etk/Bmx occurred due to TBI in vitro, and then to determine whether the expression of Etk/Bmx can be a quantification reference indicator specific for neurological injury degree of TBI. On the other hand, the method of the present invention is advantageous to discuss whether the inhibition of the expression of Etk/Bmx can obtain the effect of treating or retarding the injury degree of TBI. Further, to develop related potential detection tool associated with the treatment or retardation of TBI.

1. Animal Model of Controlled Cortical Impact (CCI):

According to the experimental designs, male Sprague-Dawley (SD) rats with weight of 200-300 g are randomly sorted into four groups, wherein rats of normal group are not treated by any surgery, and rats of the other three groups (sham, impact once and impact twice) are anesthetized with Chloral Hydrate (400 mg/kg) by an intraperitoneal injection. After the rats are deeply anesthetized, medical iodine (70 wt %) is used to locally sterilize and then the rats are placed on a stereotaxic frame. After executing a midline skin incision by a sterilized knife, a right-side craniotomy is made by drilling skull bone with high-speed drill (Nakanishi Ultimate 400) to expose the skull. Controlled cortical impact (CCI) is executed to the cortex of right-side brain with mechanical impact at 2.5 m/s once or 5 m/s twice, or without mechanical impact (i.e. sham group). After injury, the impact site is covered with a plastic skull cap, and skin is sutured. Sham rats underwent the same surgical procedure including anesthesia and craniotomy, but are not injured. Rats are sacrificed at appropriate times for experiments.

2. Western blotting:

Traumatic brain tissue samples are lysed in RIPA buffer. 50-100 μg of cell lysates are resolved into 8% to 15% SDS/PAGE gel, and then transferred onto nitrocellulose (NC) membranes. Subsequently, blots are incubated with antibodies raised against related proteins, including: anti-Etk/Bmx (Transduction Laboratories), anti-pTyr-Etk (Cell Signaling) and anti-Actin (Sigma). According to the property of the primary antibodies, secondary antibodies, such as Donkey peroxidase-conjugated anti-rabbit or anti-mouse antibodies, are used, and then enhanced chemiluminescence reagent (ECL) is finally added to develop X-ray film for analysis.

3. Reverse transcription polymerase chain reaction (RT-PCR) and Real-time polymerase chain reaction (Q-PCR):

Total RNA is extracted from traumatic brain tissue by utilizing Trizol reagent. Prior to RT-PCR, 1 μg of RNA is initially treated with DNase I (Ambion Inc., Austin, Tex.) to degrade genomic DNA. Thereafter, 50 ng of treated RNA is used for one-step RT-PCR reaction. Gene expression is quantified by Real-time PCR (Q-PCR) using SYBR Green dye. All Q-PCR reactions are performed on a platform of 7900HT ABI Sequence Detection System (Applied Biosystems). The sequences of primers are listed, as follows: GAPD forward sequence 5′-GCACCGTCAAGGCTGAGAAC-3′ and reverse sequence 5′-ATGGTGGTGAAGACGCCA-3′. The mRNA expression levels of GAPD is defined as a standard in the quantitative analyses. The forward sequence of the primer for mEtk/Bmx is 5′-CACACCACCTCAAAGATTTCATGG-3′ and the reverse sequence thereof is 5′-CATACTGCCCCTTCCACTTGC-3′.

4. Immunofluorescence Analysis:

Twenty-four hours after TBI, rats are anesthetized and perfused through the ascending aorta with 100 mL of cold normal saline, followed by 100 mL of 4% paraformaldehyde (PFA) in PBS. Entire brains are removed and post-fixed in the same fixative for 3 days, followed by dehydration using 30% sucrose for 1 week. Sections are cut at a thickness of 12 microns in a freezing microtome and stored at −20° C. For immunostaining, tissue sections are fixed with 4% PFA for 10 minutes. After several washes in PBS, the tissue sections are incubated with blocking buffer containing 0.3% Triton X-100 and 4% bovine serum albumin for 1 hour at room temperature, and are then stained with the desired primary antibody reconstituted in PBS, 2% goat serum at 4° C. for 14-16 hours. The primary antibodies of the present invention include anti-Etk/Bmx (Cell Signaling), anti-neurofilament M (NF-M, Millipore), anti-GFAP (Transduction Laboratories), these primary antibodies are diluted with the blocking buffer (the dilution ratio=1:100). After three rinses in PBS to remove the remaining primary antibodies, the tissue sections are incubated with goat anti-rabbit IgG FITC conjugate (1:100) and goat anti-mouse IgG Rhodamine conjugate (1:100) for 45 minutes at room temperature. Finally, 1 mg/mL DAPI is added into the dilution of secondary antibodies for the last 15 minutes. After washing in PBS several times, the tissue sections are mounted with Crystal Mount (Sigma) and analyzed by a Leica microscope, a SROT RTTM CCD camera or laser-scanning confocal microscope.

5. Triphenyltetrazolium Chloride (TIC) Staining:

All rats are sacrificed at 24th hours after impact injury, and the entire brain tissue is then removed, and sliced into 1.0 mm sections. The brain slices are incubated in 2 wt % triphenyltetrazolium (TTC) dissolved in Ringer's solution for 20 minutes at 37° C., and then washed by saline 3 times, followed by being transferred to 2 wt % paraformaldehyde solution for fixation. The volume of brain contusion, as revealed by negative TTC stains indicating dehydrogenase-deficient tissue, is measured in each slice and summed using computerized planimetry (Image-Pro Plus).

6. Comparison of Two Lateral Cortexes of Brain and Increasing Expression of Etk/Bmx after Brain Impact:

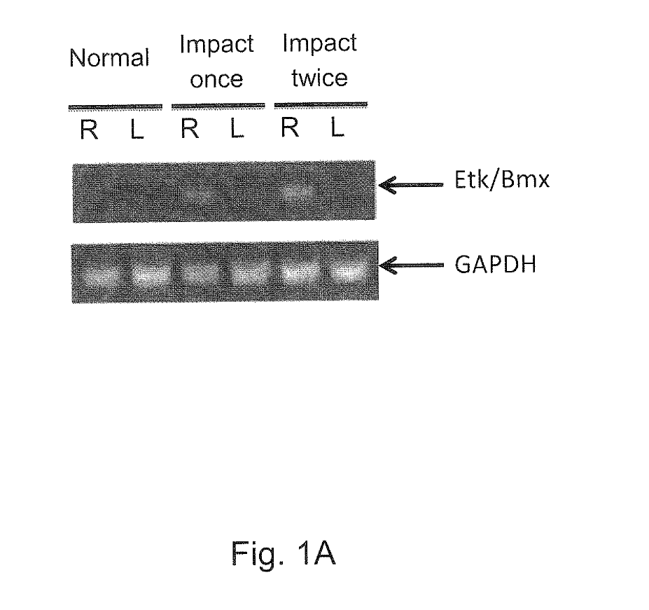

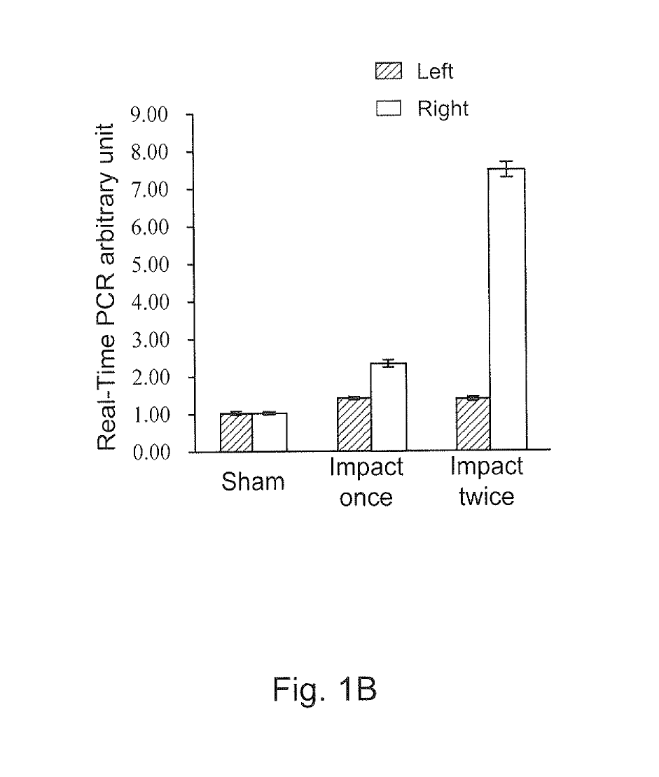

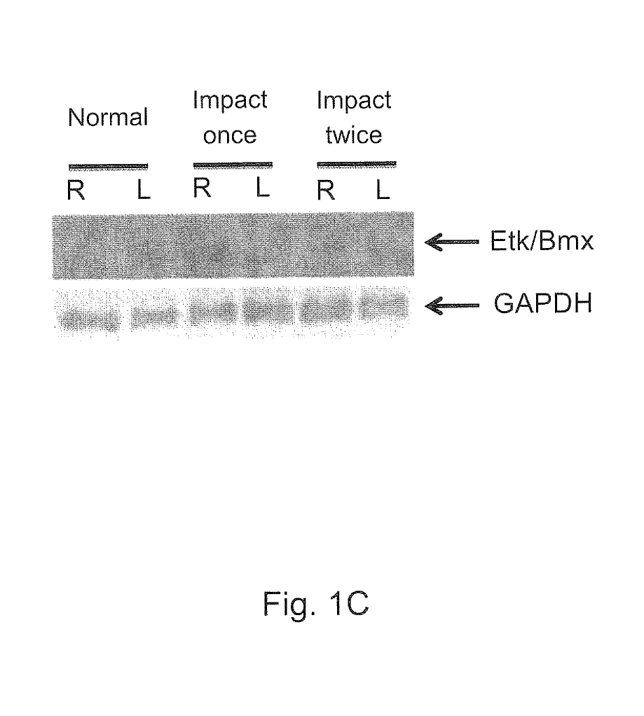

To clarify the correlation between two lateral cortexes of brain after brain impact injury occurs to one of the two lateral cortexes, SD rats are sorted into 4 groups randomly, including normal, sham, impact once (2.5 m/s) and impact twice (5 m/s), respectively. To impact the right cortex and suture it, rats are sacrificed 24 hours later. To take off brains and separate to left and right cortexes, and then to grind cortex tissues, centrifuge and sample the cell extraction for RT-PCR, Real-time PCR (Q-PCR) and Western blotting. The results are shown in FIGS. 1A, 1B and 1C, which are autoradiograms and statistic graphs of the increasing expressions of Etk/Bmx mRNA and protein of brain tissues after TBI experiments by Western blotting according to a preferred embodiment of the present invention, wherein FIGS. 1A and 1B are used to analyze the mRNA expression in two lateral cortexes when comparing the impact once or twice brain injury (2.5 m/s and 5 m/s) to the normal cortex, GAPDH is an internal control. In comparison with left cortex tissues of the normal group, the mRNA expression of Etk/Bmx of the impact once (2.5 m/s) are performed a multiple increment; and FIG. 1C shows the protein expression of Etk/Bmx within the left and the right cortexes in three groups by Western blotting.

Therefore, referring back to FIGS. 1A, 1B and 1C, in the preferred embodiment of the present invention, PCR product, Real-time PCR analysis and Western blot analysis demonstrated that Etk/Bmx is not apparently increased in expression when comparing the post-impact injury to the two lateral cortexes of normal group. Furthermore, after the right cortex is impacted once or twice, the mRNA or protein expression of Etk/Bmx of left cortex of the same rat is not apparently increased. In comparison with the left cortex, the mRNA or protein expression of Etk/Bmx of right cortex impacted once is apparently increased, and the mRNA expression of right cortex impacted twice is increased up to 7 fold.

7. Correlation Between Etk/Bmx Expression and Injury Degree of TBI, Analyzed Based on Results of Western Blotting:

To clarify the correlation between protein expression of Etk/Bmx or S100β and injury degree of trauma brain injury, rats are randomly sorted into 4 groups, including normal, sham, impact once (2.5 m/s) and impact twice (5 m/s), respectively. To impact the right cortex and suture it, rats are sacrificed 24 hours later. After brain samples are removed and separated these samples into left and right cortexes, for frozen sections or tissue grinding to obtain protein extractions for Western blotting.

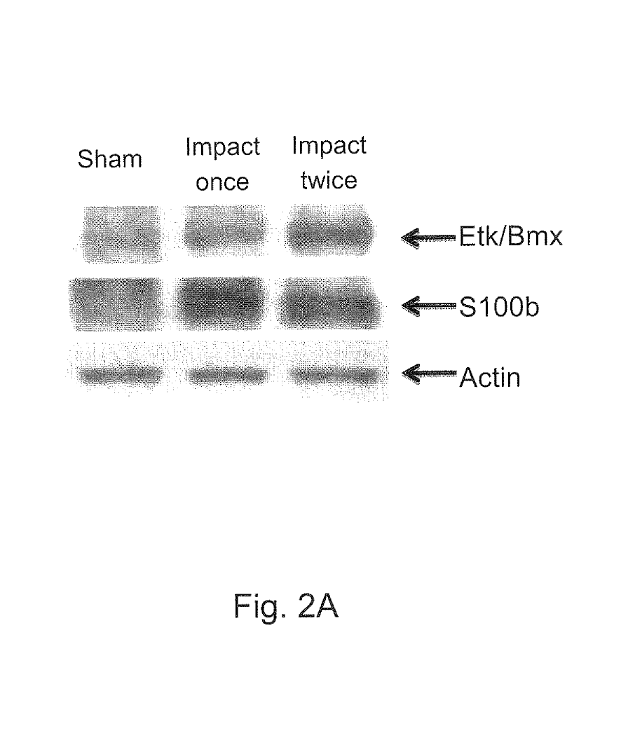

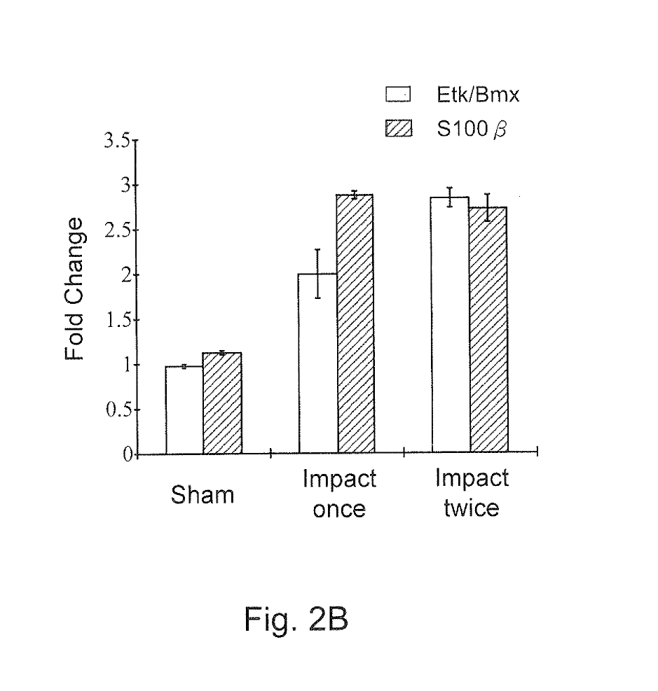

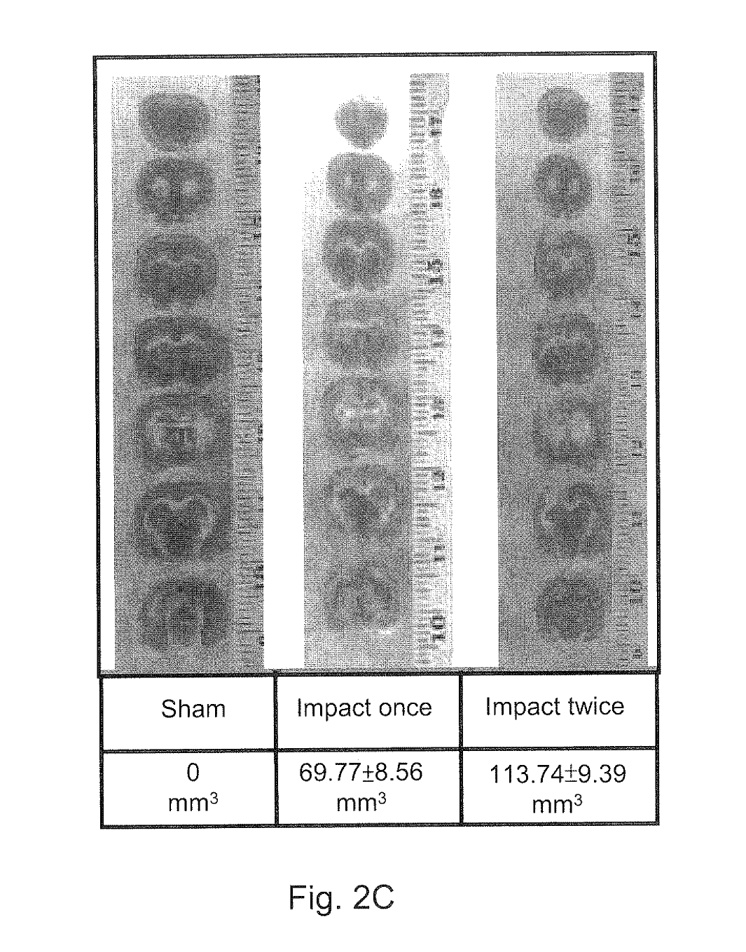

Referring now to FIGS. 2A, 2B and 2C, autoradiograms and statistic graphs of the expressions of Etk/Bmx and S100β protein of brain tissues after TBI experiments by Western blotting and frozen section according to the preferred embodiment of the present invention are shown, wherein Western blot analysis of protein expression of Etk/Bmx shown in FIGS. 2A and 2B reflects that the protein expression of Etk/Bmx is increased almost 2 fold when impact is performed once (2.5 m/s) and 2.7 fold when impact is performed twice (5 m/s). However, the protein expression of S100β is increased about 2.5 fold when impact is once and twice. Referring to FIG. 2C, the result of TTC staining reflects that the cortical injured areas are increased when impact force is increased. Therefore, to compare frozen sections of brain samples of sham group, impact once group and impact twice group by TTC (2,3,5-triphenyltetrazolium chloride) staining, it is easy to observe that the white injured areas of the impact twice group, the impact once group and the sham group are 113.74±9.39 mm², 69.77±8.56 mm² and 0 mm², respectively. Western blot analysis of protein expression of Etk/Bmx of four groups shown in FIGS. 213 and 2C reflects that the protein expression of Etk/Bmx of the impact twice group is increased almost 2.6 fold of the sham group, and that of the impact once group is increased almost 2 fold of the sham group. However, the protein expression of S100β is increased when impact is once and twice, i.e. there is no difference of protein expression of S100β between the impact once group and the impact twice group.

8. Different Time Correlation for Etk/Bmx and GFAP after TBI, Analyzed Based on Results of Western Blotting:

To clarify if Etk/Bmx and GFAP expresses with respect to different time (such as 1 hr, 3 hrs, 6 hrs, 1 day, 4 days and 7 days) after trauma brain injury, SD rats are sorted into 3 groups randomly, including sham, impact once (2.5 m/s) and impact twice (5 m/s), respectively. To impact the right cortex and suture it, rats are sacrificed in different time after impact (such as 1 hr, 3 hr, 6 hr, 1 d, 4 d and 7 d). After brain samples are removed, the brain samples are ground, centrifuged and sampled the cell extractions for Western blot assay.

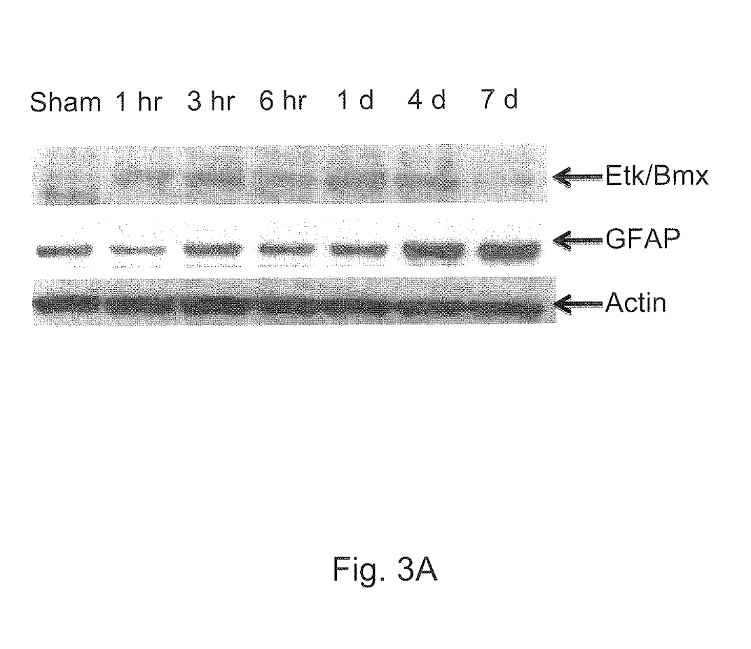

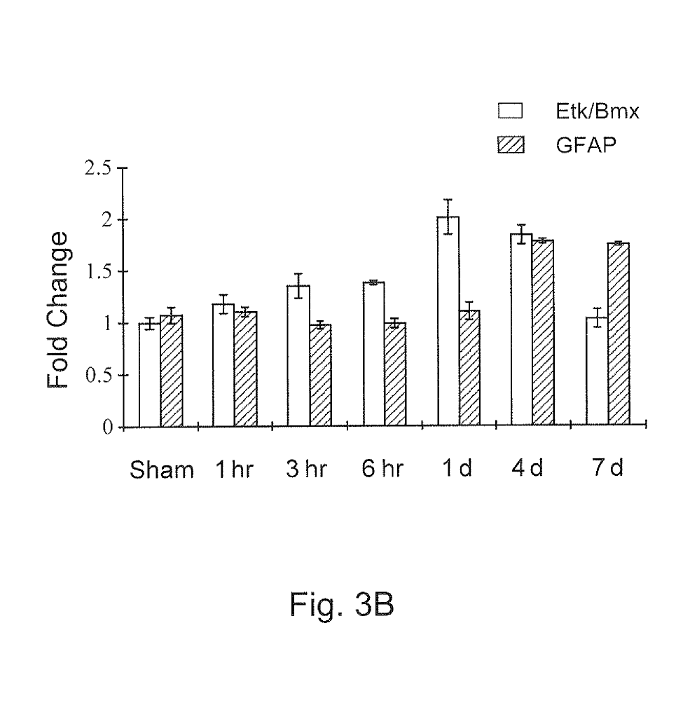

Referring now to FIGS. 3A and 3B, autoradiograms and statistic graphs of the expressions of Etk/Bmx and GFAP protein of brain tissues in different time after TBI experiments by Western blotting according to the preferred embodiment of the present invention are shown, wherein FIG. 3A is used to analyze the protein expression of Etk/Bmx and GFAP when impact is once (2.5 m/s) and then sacrificed in different time after impact (such as 1 hr, 3 hr, 6 hr, 1 d, 4 d and 7 d); the statistic graph of FIG. 3B shows that the protein expression of Etk/Bmx of the impact once group (2.5 m/s) is significantly increased 3 hour later after impact, and the protein expression of GFAP is obvious increased 4 days later after impact. Therefore, in the preferred embodiment of the present invention, the expression of Etk/Bmx and GFAP is increased with respect to time after brain impact. The protein expression of GFAP is increased 4 days later after impact (at a later stage) and still lasts a high expression degree up to 7 days after impact. In contrast, the protein expression of Etk/Bmx appears significantly 1 day later after injury and continues until 4 days later after injury, and then returns to normal level 7 days later after injury.

9. Different Expression Locations of Etk/Bmx and GFAP in Brain Tissues, Analyzed by Immunofluorescence Analysis:

To clarify the distribution locations of Etk/Bmx and GFAP in brain tissues after impact with respect to injury site, SD rats are sorted into 2 groups randomly, such as sham and impact once (2.5 m/s), respectively. To impact the right cortex of the impact once group and suture it, rats are sacrificed 24 hours later. After brain samples are removed for frozen sections and then observed these slices by immunofluorescence analysis.

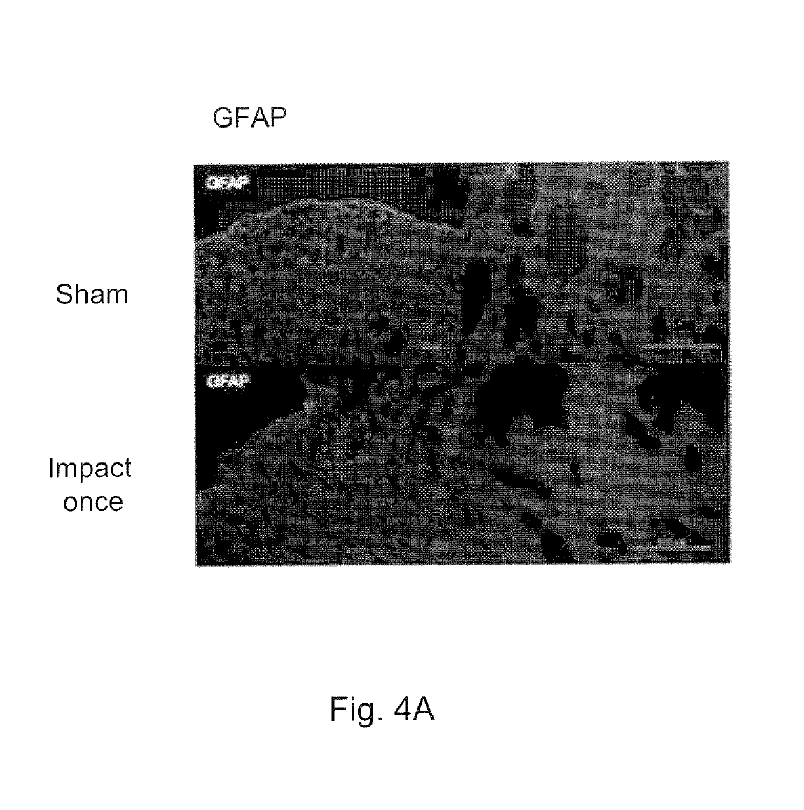

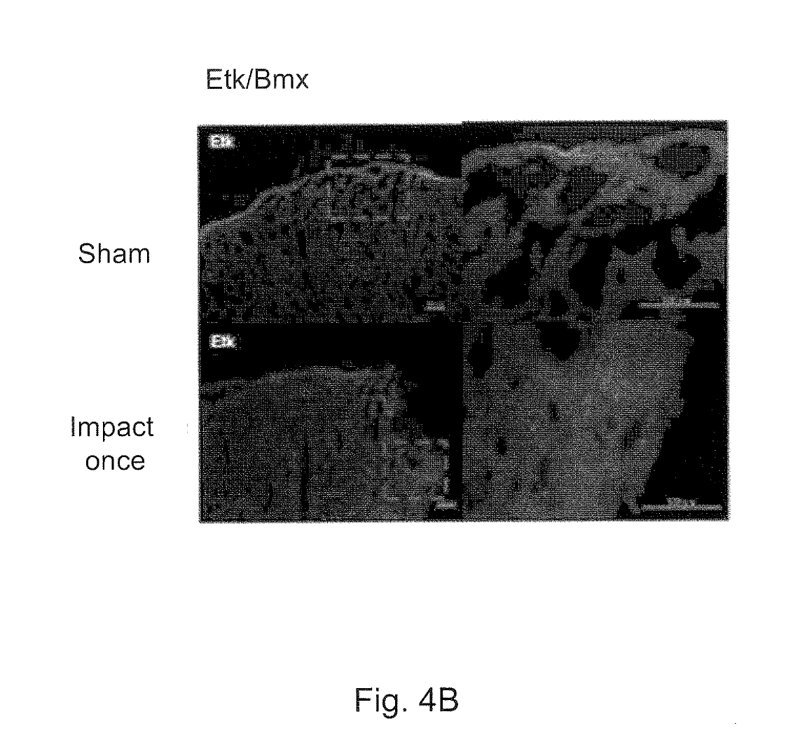

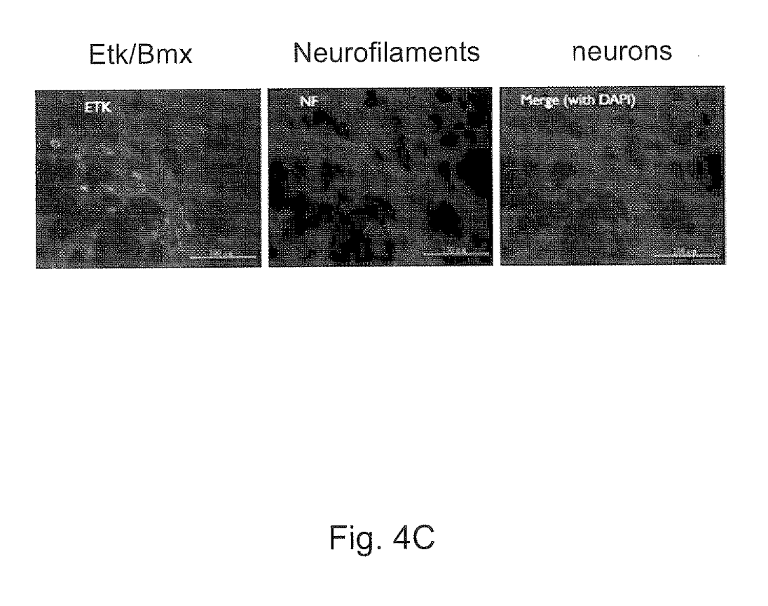

Referring now to FIGS. 4A, 4B and 4C, photographs of frozen section of brain tissues after TBI experiments by immunofluorescent staining to the preferred embodiment of the present invention are shown, wherein FIGS. 4A and 48 are used to show the expression locations of Etk/Bmx and GFAP in the brain tissues of the frozen sections by immunofluorescent staining, wherein expressions of two types of fluorescence markers (green) of Etk/Bmx and GFAP can be found around the impacted tissue, wherein the expression level is decreased as distance away from the injury site increases. Referring to FIG. 4C, the location of fluorescence markers (green) of Etk/Bmx is overlapped with the location of fluorescence markers (red) of neurofilament immunostaining to form an overlapped fluorescence image (i.e. blue).

In details, as shown in FIGS. 4A and 4B, after the frozen sections of the brain tissue of the impact once group is stained by immunofluorescence analysis, a fluorescence microscopy can observe that fluorescence signals increase at the brain tissues near the injury site, i.e. Etk/Bmx and GFAP are distributed at the brain tissues near the injury site, wherein the fluorescence signal is decreased as distance away from the injury site increases. As shown in FIG. 4C, the fluorescence microscopy can observe that Etk/Bmx is stained with green fluorescence, and the neurofilament is stained with red fluorescence, wherein the overlapped (i.e. merged) image has blue fluorescence. Thus, these fluorescence signals observed at the injury site is come from neurons.

10. Different Time Correlation for Expressions of Etk/Bmx and Trauma/Inflammatory Markers (but No Time Correlation for Expressions of Other Tyrosine Kinase-Related Signal Transmission Molecules), Analyzed by Western Blotting:

To clarify if other signal transmission molecules express with respect to time (such as 1 hr, 3 hr, 6 hr, 1 d, 4 d and 7 d) after trauma brain injury, SD rats are sorted randomly to two groups, including sham and impact once (2.5 m/s), respectively, for Western blotting. To impact the right cortex of the impact once group and suture it, rats are sacrificed in different time (such as 1 hi, 3 hr, 6 hr, 1 d, 4 d and 7 d) after impact. After brain samples are removed, the brain samples are ground, centrifuged and sampled protein extractions for Western blot assay, wherein protein extractions are hybridized by primary antibodies of Etk/Bmx, Tec, Btk, Src, Fak, Stat3, Bcl2 or LC3, respectively, and then incubated to their specific secondary antibody for detecting their protein expressions.

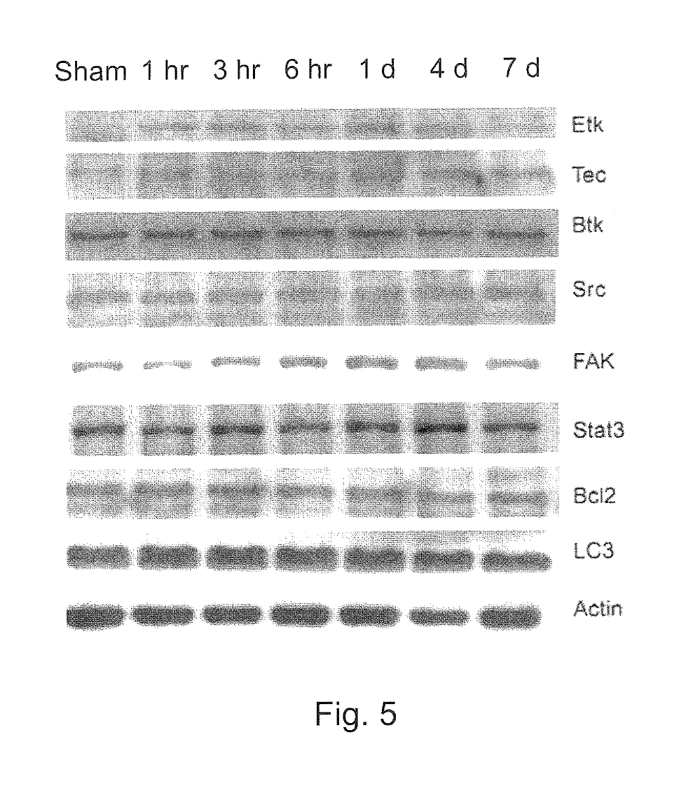

Referring now to FIG. 5, an autoradiogram of various protein expressions of brain tissues after TBI experiments by Western blotting according to the preferred embodiment of the present invention is illustrated, wherein tyrosine kinase families and apoptosis-related marker molecules in brain tissues are expressed at different time points, and only the protein expression of Etk/Bmx is increased after TBI, i.e. After impact, only the protein expression of Etk/Bmx is apparently varied at different time points, but the protein expression of other tyrosine kinase-related signal transmission molecules, such as Tec, Btk, Src, FAK, Stat 3, Bcl2 and LC3, in brain tissue of the impact once groups and the sham group all appear insignificant variation after trauma, even in different sacrificed time (1 hr, 3 hr, 6 hr, id, 4 d and 7 d). Therefore, the traumatic brain injury (TBI) can not widely cause the increase of the protein expression of all tyrosine kinase-related signal transmission molecules, but only can affect specific biomarker molecules, such as Etk/Bmx.

As described above, the method for detecting and examining traumatic brain injury (TBI) in vitro according to the present invention firstly obtains a sample separated from a traumatic brain tissue of an animal (such as human) after the brain is impacted by a traumatic brain injury, and the sample is examined in vitro for detecting an expression of Etk/Bmx protein of the sample expressed due to TBI, wherein the expression of Etk/Bmx protein is defined as a quantification reference indicator of TBI injury degree for examining and evaluating a neurological injury degree of TBI due to the external impact, wherein the sample can be brain tissue, cerebrospinal fluid or blood serum, which is sampled from the animal, such as brain tissue, cerebrospinal fluid, blood serum or other samples naturally removed or separated from the animal. The purpose of the method according to the present invention is not to directly obtain the diagnosis result of neurological injury of TBI, but is to obtain related information (i.e. the expression of Etk/Bmx protein) as an intermediate result, so as to provide a quantification reference indicator of TBI injury degree for examining and evaluating a neurological injury degree of TBI due to the external impact.

The present invention has been described with a preferred embodiment thereof and it is understood that many changes and modifications to the described embodiment can be carried out without departing from the scope and the spirit of the invention that is intended to be limited only by the appended claims.

What is claimed is:

1. A method for detecting and examining traumatic brain injury (TBI) in vitro, comprising steps of:
(a) sampling a sample from a traumatic brain tissue of an animal after occurring TBI due to an external impact, wherein the external impact is a closed-type TBI or a punctured-type TBI;
(b) examining the sample for detecting an expression of Etk/Bmx mRNA expressed due to TBI, wherein reverse transcription polymerase chain reaction (RT-PCR) or real-time polymerase chain reaction (Q-PCR) is used for examining the sample and includes steps of: (1) extracting total RNA from the traumatic brain tissue by utilizing Trizol reagent; (2) treating 1 μg of the RNA with DNase I to degrade genomic DNA therein; (3) using 50 ng of the treated RNA for one-step RT-PCR or Q-PCR, so as to detect the expression of Etk/Bmx mRNA expressed due to TBI, wherein a forward sequence of a primer for the Etk/Bmx mRNA is 5′-CACACCACCTCAAAGATTTCATGG-3′, and a reverse sequence thereof is 5′-CATACTGCCCCTTCCACTTGC-3′; and wherein a mRNA expression level of GAPD is defined as a standard in a quantitative analysis, a forward sequence of a primer for GAPD is 5′-GCACCGTCAAGGCTGAGAAC-3′, a reverse sequence thereof is 5′-ATGGTGGTGAAGACGCCA-3′, and the expression of Etk/Bmx mRNA is quantified by the Q-PCR using SYBR Green dye; and
(c) defining the expression of Etk/Bmx mRNA as a quantification reference indicator of TBI injury degree for examining and evaluating a neurological injury degree of TBI due to the external impact.
